(19) United States
(12) Patent Application Publication
(10) Pub. No.: US 2017/0273876 A1
(43) Pub. Date: Sep. 28, 2017

(54) BENEFIT DELIVERY PARTICLE

(71) Applicant: Conopco, Inc., d/b/a UNILEVER, Englewood Cliffs, NJ (US)

(72) Inventors: Ralph CALVERT, Wirral (GB); Joanne Louise COOK, Eastham, Wirral (GB); Richard Mackenzie READ, Tarporley (GB); Glyn ROBERTS, Wirral (GB); Stephen Lee WIRE, Higher Bebington, Wirral (GB)

(73) Assignee: Conopco, Inc., d/b/a UNILEVER, Englewood Cliffs, NJ (US)

(21) Appl. No.: 15/505,211

(22) PCT Filed: Aug. 28, 2015
(86) PCT No.: PCT/EP2015/069803

§ 371 Date: Feb. 20, 2017

(30) Foreign Application Priority Data
Sep. 3, 2014 (EP) 14183460.6

(51) Int. Cl. A61K 8/02; A61K 8/25; A61Q 5/12; A61K 8/73

(52) U.S. Cl. CPC A61K 8/0283; A61K 8/73; A61K 8/25; A61Q 5/12; A61Q 19/00

(57) ABSTRACT

A hydrogel particle, comprising: a) a matrix, and b) a silicone or blend or silicones, wherein the silicone or blend of silicones has an average molecular weight of greater than 400 Da, and wherein the particle has an average diameter of 0.1 to 15 mm and wherein the silicone is present in an amount of from 5 to 40 wt %, preferably from 25 to 40 wt % by total weight of the particle.

### TECHNICAL FIELD

The present invention relates to a hydrogel particle, comprising a matrix and a silicone or blend of silicones, and to methods of using such particles. These particles are useful in a wide range of home and personal care compositions, with particular relevance to compositions that are used in the treatment of hair and skin.

### BACKGROUND

Many home and personal care formulations seek to deliver silicones to substrates, for example, fabric, hair and skin.

Hydrogels can be useful in the formulation of home and personal care products. Hydrogels are typically formed by gelation/precipitation of biodegradable polymer(s) with appropriate ions or reagents or by pH shift or temperature change. The hydrogels can be reduced to particles by a variety of methods, for example extrusion followed by chopping up into particles, or breaking up by the use of rotary blades or paddle stirrers.

Various benefit materials have been incorporated into hydrogels, such as preservatives, pigments, opacifiers amongst others. These materials can then be delivered to a substrate by application of particles.

WO 2012/154505 (Johnson & Johnson) discloses a composition comprising aqueous carrier, hydrogel particles comprising saccharides, and surfactants and wherein the composition does not show any phase separation over a period of ten days at room temperature as measured using a defined Phase Stability Test.

EP 1 072 259B (Shiseido) discloses a microcapsule comprising an inner oil phase and a water phase, there being dispersed in an outer oil phase, wherein the inner oil phase is an oil droplet having an average particle size of from 0.01 to 3 micron, the oil droplet is encapsulated in the water phase, the water phase comprising a capsulating agent being a hydrophilic polymer gelling agent, wherein the polymer gelling agent essentially is agar or carrageenan, and wherein the microcapsule is dispersed in the outer oil phase.

KR2007-084762 (LG Household and Healthcare) discloses a hair cosmetic composition for conditioning comprising a reactive silicone capsule.

We have found, however, that where silicones are carried by hydrogel particles, delivery to the substrate is low and as such little or no consumer benefit can be perceived.

We have now found that improved hydrogel particles can be provided that contain a silicone, or blend of silicones with an average molecular weight of 400 Da or greater. This results in increased silicone deposition to the substrate thus effecting a consumer perceivable benefit.

### BRIEF DESCRIPTION OF THE INVENTION

In a first aspect, the invention provides a hydrogel particle, comprising

a) a matrix, and

b) a silicone or blend or silicones,

wherein the silicone or blend of silicones has an average molecular weight of greater than 400 Da, and wherein the particle has an average diameter of 0.1 to 15 mm and wherein the silicone is present in an amount of from 5 to 40 wt %, preferably from 25 to 40 wt % by total weight of the particle.

A second aspect of the present invention provides a method of treatment of a substrate, preferably wherein the substrate is selected from skin and hair, which includes the step of contacting the substrate with the particles according to the first aspect of the invention, and the step of applying a releasing force to the hydrogel particle to release the silicone.

### DETAILED DESCRIPTION OF THE INVENTION

The Particle

The particles of the invention are hydrogel particles, which contain a matrix and a silicone. They are preferably spherical, or essentially spherical.

Typically, the particle has an average diameter of 0.1 to 15 mm, for example, 0.1 to 8, preferably from 0.2 to 10 mm, more preferably from 0.5 to 8 mm and most preferably 0.5 to 4 mm.

The particle can be suspended in a salt solution, preferably calcium chloride solution, in order to improve its robustness. This is particularly useful for storage and transport.

The Matrix

The matrix may be formed by gelation or precipitation of biodegradable polymer(s) either with appropriate salts or reagents or by pH shift or temperature change. Preferred polymers include alginate, chitosan, cellulose, karageenan, other gums and mixtures thereof, most preferably karageenan and alginate.

Most preferably, the particles are formed chondrus crispus (karageenan) and alginate with calcium chloride.

In addition, the matrix can contain further additives including dyes, pigments, micas, emotives, pearlisers, opacifiers, minerals, oils, preservatives, proteins, amino acids and mixtures thereof, preferably selected from proteins and amino acids.

The Silicone

The hydrogel particle comprises at least one silicone with an average molecular weight of greater than 400 Da. Where it contains more than one silicone, herein called a blend of silicones, the overall average molecular weight of the blend of silicones is greater than 400 Da.

Crosslinked silicones may be used, including silicone gums.

Preferably the silicone blend contains less than 50% (by weight) of silicone having a molecular weight of less than 400 Da, more preferably less than 30%, even more preferably less than 10% and most preferably less than 5%.

More preferably, the silicone comprises a blend of silicones, which comprises one part with a molecular weight of less than 10 kDa and one part with a molecular weight of greater than 10 kDa. Preferably, said part having a molecular weight of less than 10 kDa is present at less than 50% by weight of the total silicone, more preferably less than 20% by weight of the total silicone.

Examples of suitable silicones are available from Dow Corning under the following tradenames:—XIAMETER® PMX-1413 FLUID, XIAMETER® PMX-1503 FLUID, XIAMETER® PMX-200 SILICONE FLUID 1,000CS, XIAMETER® PMX-200 SILICONE FLUID 10,000CS, XIAMETER® PMX-200 SILICONE FLUID 100,000CS, XIAMETER® PMX-200 SILICONE FLUID 100CS, XIAMETER® PMX-200 SILICONE FLUID 12,500CS, XIAMETER® PMX-200 SILICONE FLUID 2,000CS, XIAMETER® PMX-200 SILICONE FLUID 200CS, XIAMETER® PMX-200 SILICONE FLUID 20CS, XIAMETER® PMX-200 SILICONE FLUID 30,000CS, XIAMETER® PMX-200 SILICONE FLUID 300,000CS, XIAMETER® PMX-200 SILICONE FLUID 350CS, XIAMETER® PMX-200 SILICONE FLUID 500,000CS, XIAMETER® PMX-200 SILICONE FLUID 500CS, XIAMETER® PMX-200 SILICONE FLUID 50CS, XIAMETER® PMX-200 SILICONE FLUID 5CS, XIAMETER® PMX-200 SILICONE FLUID 60,000CS, XIAMETER® PMX-9027 FLUID and XIAMETER® PMX-1403 FLUID.

The hydrogel particle comprises from 5 to 40 wt %, preferably from 25 to 40 wt %, most preferably from 30 to 40 wt % by total weight of the particle, of silicone.

Suitable hydrogel beads containing silicone oil can be obtained from GeniaLab BioTechnologie Produkte and Dienstleistungen GmbH, Germany, under the tradename ISP Captivates GL 7782. The beads may be prepared by the method described in U.S. Pat. No. 6,467,699, and the silicone incorporated therein.

Particles in Compositions

The particles of the invention may be incorporated into compositions that are used in the treatment of surfaces, particularly hair and skin.

A personal care composition comprising at least one particle according to the invention is advantageously a deodorant, antiperspirant, shampoo, hair conditioner or skin care or skin cleansing product. hair conditioner, serum or skin moisturiser.

Such compositions may contain benefit agents. Preferred examples include flavours and fragrances, conditioning agents (for example water-insoluble quaternary ammonium materials and/or silicones), sunscreens, colour protection agents, ceramides, antioxidants, dyes, lubricants, unsaturated oils, emollients/moisturiser, insect repellents and/or antimicrobial agents.

For skin compositions the preferred benefit agents include one or more of fragrances, sunscreens, skin lightening agents, antimicrobials, oils and insect repellents. For hair compositions the list of preferred benefit agents is the same with the addition of colour protection agents and dyes.

Preferred antimicrobials include Triclosan™, climbazole, octapyrox, ketoconizole, zinc pyrithione, and quaternary ammonium compounds.

Preferred sunscreens and/or skin lightening agents are vitamin B3 compounds. Suitable vitamin B3 compounds are selected from niacin, niacinamide, nicotinyl alcohol, or derivatives or salts thereof. Other vitamins which act as skin lightening agents can be advantageously included in the skin lightening composition to provide for additional skin lightening effects. These include vitamin B6, vitamin C, vitamin A or their precursors. Mixtures of the vitamins can also be employed in the composition of the invention. An especially preferred additional vitamin is vitamin B6. Other non-limiting examples of skin lightening agents useful herein include adapalene, aloe extract, ammonium lactate, arbutin, azelaic acid, butyl hydroxy anisole, butyl hydroxy toluene, citrate esters, deoxyarbutin, 1,3 diphenyl propane derivatives, 2,5 dihydroxyl benzoic acid and its derivatives, 2-(4-acetoxyphenyl)-1,3 dithane, 2-(4-Hydroxylphenyl)-1,3 dithane, ellagic acid, gluco pyranosyl-1-ascorbate, gluconic acid, glycolic acid, green tea extract, 4-Hydroxy-5-methyl-3[2H]-furanone, hydroquinone, 4 hydroxyanisole and its derivatives, 4-hydroxy benzoic acid derivatives, hydroxycaprylic acid, inositol ascorbate, kojic acid, lactic acid, lemon extract, linoleic acid, magnesium ascorbyl phosphate, 5-octanoyl salicylic acid, 2,4 resorcinol derivatives, 3,5 resorcinol derivatives, salicylic acid, 3,4,5 trihydroxybenzyl derivatives, and mixtures thereof. Preferred sunscreens useful in the present invention are 2-ethylhexyl-p-methoxycinnamate, butyl methoxy dibenzoylmethane, 2-hydroxy-4-methoxybenzophenone, octyl dimethyl-p-aminobenzoic acid and mixtures thereof. Particularly preferred sunscreen is chosen from 2-ethyl hexyl-p-methoxycinnamate, 4,-t-butyl-4′-methoxydibenzoyl-methane or mixtures thereof. Other conventional sunscreen agents that are suitable for use in the skin lightening composition of the invention include 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl-p-aminobenzoate, 3,3,5-trimethylcyclohexyl-salicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonic benzoxazoic acid and mixtures of these compounds.

Preferred anti-oxidants include vitamin E, retinol, antioxiants based on hydroxytoluene such as Irganox™ or commercially available antioxidants such as the Trollox™ series.

Perfume and fragrance materials (which include pro-fragrances) are a particularly preferred benefit agent.

The pro-fragrance can, for example, be a food lipid. Food lipids typically contain structural units with pronounced hydrophobicity. The majority of lipids are derived from fatty acids. In these ‘acyl’ lipids the fatty acids are predominantly present as esters and include mono-, di-, triacyl glycerols, phospholipids, glycolipids, diol lipids, waxes, sterol esters and tocopherols. In their natural state, plant lipids comprise antioxidants to prevent their oxidation. While these may be at least in part removed during the isolation of oils from plants some antioxidants may remain. These antioxidants can be pro-fragrances. In particular, the carotenoids and related compounds including vitamin A, retinol, retinal, retinoic acid and provitamin A are capable of being converted into fragrant species including the ionones, damascones and damscenones. Preferred pro-fragrance food lipids include olive oil, palm oil, canola oil, squalene, sunflower seed oil, wheat germ oil, almond oil, coconut oil, grape seed oil, rapeseed oil, castor oil, corn oil, cottonseed oil, safflower oil, groundnut oil, poppy seed oil, palm kernel oil, rice bran oil, sesame oil, soybean oil, pumpkin seed oil, jojoba oil and mustard seed oil.

Useful components of the perfume include materials of both natural and synthetic origin. They include single compounds and mixtures. Specific examples of such components may be found in the current literature, e.g., in Fenaroli's Handbook of Flavour Ingredients, 1975, CRC Press; Synthetic Food Adjuncts, 1947 by M. B. Jacobs, edited by Van Nostrand; or Perfume and Flavour Chemicals by S. Arctander 1969, Montclair, N.J. (USA). These substances are well known to the person skilled in the art of perfuming, flavouring, and/or aromatizing consumer products, i.e., of imparting an odour and/or a flavour or taste to a consumer product traditionally perfumed or flavoured, or of modifying the odour and/or taste of said consumer product.

Method of Treatment

The particles of the invention may be used in a method of treatment of a substrate, which includes the step of contacting the substrate with the particles of the invention, and the step of applying a releasing force to the hydrogel particle to release the silicone.

The releasing force is preferably selected from shear force, pressure and friction, preferably shear force.

The step of applying a releasing force to the hydrogel particle to release the silicone may be performed before or after application of the particle to the substrate.

Preferably, the substrate is select from skin and hair.

### EXAMPLES

### Example 1

Hydrogel Beads B1, In Accordance with the Invention, and Comparative Beads BA

Hydrogel beads were obtained from GeniaLab BioTechnologie Produkte and Dienstleistungen GmbH, Germany. The beads had a diameter of approximately 2.5 mm. Two types of beads were used:
1) beads comprising 79 wt % dimethicone, 20 wt % dimethiconol, and 1 wt % cyclotetrasiloxane, by total wt of silicone (ISP Captivates GL 7782); designated B1, and p0 2) beads that comprised silicone comprised of 85 wt % cyclopentasiloxane and 15 wt % dimethiconol by total wt of silicone; designated herein BA.

### Example 2

Treatment of Hair with Hydrogel Beads B1 and BA

Hair was treated with beads B1 and BA.

The hair was contacted with the beads and rubbed to release the silicone.

The texture of the hair was then evaluated by friction measurement. It was found that hair treated with B1 showed a significant decrease in friction, whereas treatment with BA gave no improvement versus shampoo and conditioner alone.

1. A hydrogel particle, comprising
a) a matrix, and
b) a silicone or blend or silicones,
wherein the silicone or blend of silicones has an average molecular weight of greater than 400 Da, and wherein the particle has an average diameter of 0.1 to 15 mm and wherein the silicone is present in an amount of from 5 to 40 wt %, preferably from 25 to 40 wt % by total weight of the particle.

2. A particle as claimed in claim 1, wherein the silicone blend contains less than 50% by weight of silicone having a molecular weight of less than 400 Da.

3. A particle as claimed in claim 1, wherein the blend of silicones comprises one part with a molecular weight of less than 10 kDa and one part with a molecular weight of greater than 10 kDa.

4. A particle as claimed in claim 3, wherein the part having a molecular weight of less than 10 kDa is present at less than 50% by weight of the total silicone.

5. A particle as claimed in claim 1, wherein the silicone is present in an amount of from 30 to 40 wt % by total weight of the particle.

6. A particle as claimed in claim 1, wherein the matrix comprises calcium alginate.

7. A particle as claimed in claim 1, wherein the particle further comprises an additional benefit material selected from proteins and amino acids.

8. A particle as claimed in claim 1, wherein the particle has an average particle diameter of 0.1 to 7, more preferably 0.1 to 4 mm.

9. A particle as claimed in claim 1, which is suspended in an aqueous calcium chloride solution.

10. A method of treatment of a substrate, which includes the step of contacting the substrate with the particles as defined in claim 1, and the step of applying a releasing force to the hydrogel particle to release the silicone.

11. A method as claimed in claim 19, wherein the releasing is selected from shear force, pressure and friction.

12. A method as claimed in claim 10, wherein the step of applying a releasing force to the hydrogel particle to release the silicone is performed before application to the substrate.

13. A method as claimed in claim 10, wherein the substrate is selected from skin and hair.
